(19) United States
(12) Patent Application Publication
(10) Pub. No.: US 2003/0212293 A1
(43) Pub. Date: Nov. 13, 2003

(54) Method for transferring amino acid into ketone acid(ester)

(76) Inventor: Chung-Jang Chang, Nantou City (TW)

Correspondence Address: CHARLES E. BAXLEY, ESQUIRE, Hart, Baxley, Daniels & Holton, 5 Fl., 59 John St., New York, NY, 10038, US

(21) Appl. No.: 10/141,728

(22) Filed: May 10, 2002

(51) Int. Cl. C07C 69/76; C07C 69/95; C07C 69/66; C07C 59/76

(52) U.S. Cl. 560/51; 560/55; 560/179; 562/460; 562/466; 562/567

(57) ABSTRACT

A method for transferring amino acid into ketone acid (ester) may obtain ketone acid (ester) at low cost. The method uses sodium hypochlorous acid as oxidizing agent and proceeds oxidation reaction with amino acid or its derivatives so as to obtain ketone acid and its derivatives. The sodium hypochlorous acid is easily to get with low cost and the conditions of the reaction are mild so that the method meets the needs of the industry.

### FIELD OF THE INVENTION

The present invention relates to a method for transferring amino acid into ketone acid (ester), and more particularly, the ketone acid (ester) and the derivatives thereof are obtained by oxidation reaction of oxidizing agent and the amino acid and its derivatives.

### BACKGROUND OF THE INVENTION

The medicines for curing the problems of circulation organs are increased gradually because of the number of aged people increase, the medicines for reducing blood pressure are especially requested in the market. ACE Inhibitor occupies most of the market for the medicines for reducing the blood pressure.

ACE Inhibitors, as shown in FIG. 1 includes more than two optical centers which are a combination of three amino acids, wherein as shown in the drawing, the Non-natural Hyper Phenylalanine is located beside A part and the double amino acid is located beside B part or its derivatives. Generally, the optical center of B part comes from natural amino acid which can be obtained easily by the present techniques. The L-Homophenylalanine of the A part is not a non-natural amino acid so that it has to be obtained by way of asymmetric synthesis technique and which has high degree of difficulty.

The preferable way to combine the ACE inhibitors is to proceed a asymmetric reduction reaction to the EOPB1 to obtain α-Hydroxycarboxylic Acid Ester ((R)-EHPB22) of R form. As shown in FIG. 2, the compound 22 is the initial product and the hydroxide is transferred to be the leaving group by Trflate, and then proceed a substitution reaction with the amino acid on the other side to obtain the Enalapril. During the substitution process, no racemization products are generated. The EHPB22 of the R form can be completely transferred into the ACE inhibitors of S form. The technique can be used for all the composing of transferring ACE Inhibitors. The advantage of this technique is the high optics selectivity which is a potential method and the newest trend for composing ACE inhibitors.

It is understood that the (R)-EHPB22 is an important mediate portion of for composing various types of ACE inhibitor. The EOPB1 may also be composed into various types of ACE inhibitor. In the present technique, the composing of EOPB1 includes the following three methods as shown in FIG. 3, which are:

The first one is to use 2-phenyl ethyl bromide 13 as an initial product which is reacted with Diethyl Oxalate 15. In this composing process, the 2-phenyl ethyl magnesium bromide 14 is obtained by Grignard reaction and is reacted with the Diethyl Oxalate 15. It is to be noted that the absorbing power is only 40% because by-product (the dimmer of 13) is generated. The product has to be evaporated by decompression distillation so that it is difficult to purify. Therefore, it is not a proper way to industrialize.

The second method is to proceed an oxidization processes to α-Hydroxycarboxylic Acid Ester 16 to obtain EOPB 22. In this method, four equivalent weights of oxidizing agent are used so that it is difficult to purify. Besides, some oxidizing agents are special which are costly.

The third method is to proceed an esterification reaction to α-keto-carboxylic Acid 17 and the shortcoming of this method is that the initial product α-keto-carboxylic Acid 17 is difficult to get and involves too much cost.

The three methods mentioned above are not suitable to be industrialized production. In other words, there is no method to generate EOPB1 or its derivatives (R)-EHPB22 with high purification, high production rate and low cast. The present technique cannot meet the requirements in the markets.

The present invention intends to provide a method for transferring amino acid into ketone acid (ester), and the method uses sodium hypochlorous acid as oxidizing agent and proceeds oxidation reaction with amino acid or its derivatives so as to obtain ketone acid and its derivatives. The sodium hypochlorous acid is easily to get with low cost and the conditions of the reaction are mild so that the method meets the needs of the industry.

### SUMMARY OF THE INVENTION

The present invention intends to provide a method for transferring amino acid into ketone acid (ester), and the method uses sodium hypochlorous acid as oxidizing agent and proceeds oxidation reaction with amino acid or its derivatives so as to obtain ketone acid and its derivatives. The sodium hypochlorous acid is easily to get with low cost and the conditions of the reaction are mild so that the method meets the needs of the industry.

The present invention will become more obvious from the following description when taken in connection with the accompanying drawings which show, for purposes of illustration only, a preferred embodiment in accordance with the present invention.

### BRIEF DESCRIPTION OF THE DRAWINGS

FIG. 1 shows the structure of the ACE Inhibitors;

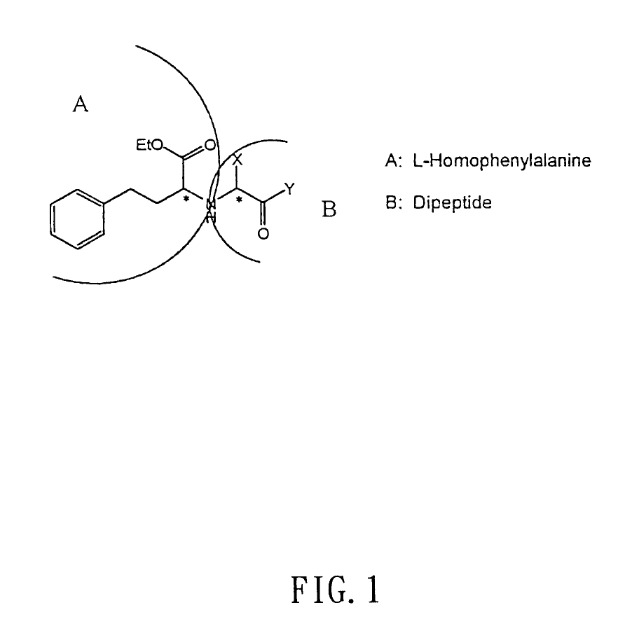

FIG. 2 shows the preferred conventional way to compose the ACE Inhibitors;

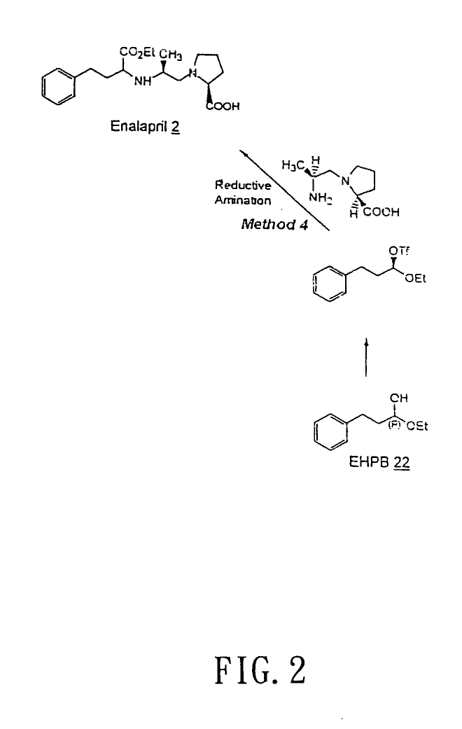

FIG. 3 shows three preferred conventional ways to compose the EOPB1;

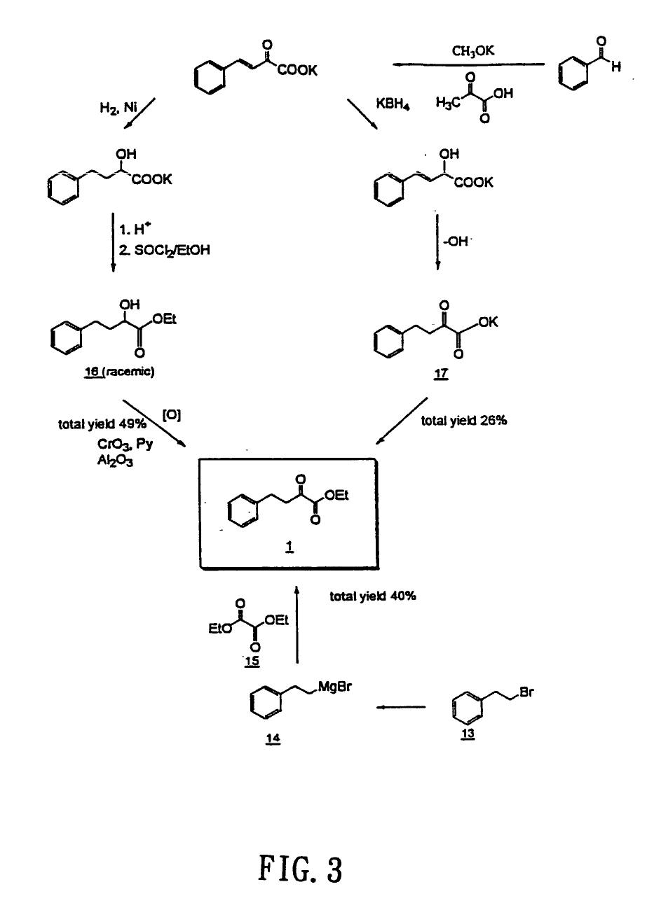

FIG. 4 shows the structure of the steps of the conventional method of oxidation action of oxidization agent and amino acid;

FIG. 5 shows the reaction of the present invention;

FIG. 6 shows the structure of the reaction of an embodiment of the present invention;

FIG. 7 shows the structure of the amino acid oxidation reaction of the present invention, and

FIG. 8 shows the structure of (R)-EHPB22 obtained by asymmetric reduction reaction.

### DETAILED DESCRIPTION OF THE PREFERRED EMBODIMENTS

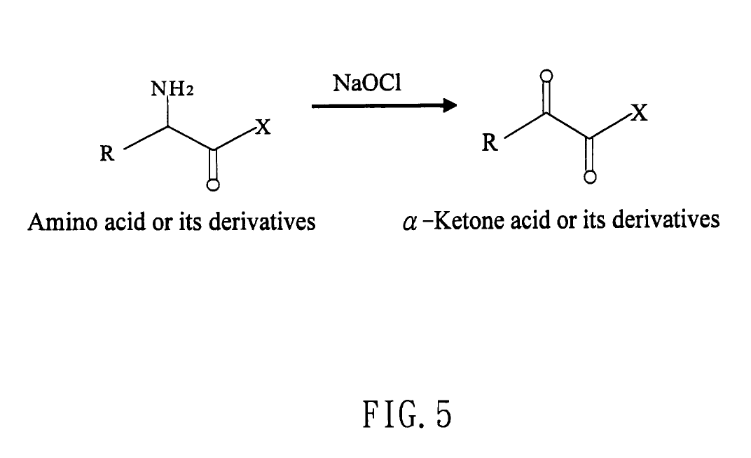

Referring to FIG. 5, the present invention provides a method for transferring amino acid into ketone acid (ester). The amino acid or its derivatives is used as initial product and take sodium hypochlorous acid to proceed oxidation reaction with amino acid or its derivatives so as to obtain ketone acid and its derivatives. The reaction is shown in FIG. 5 and the R and X in the drawing are substituents.

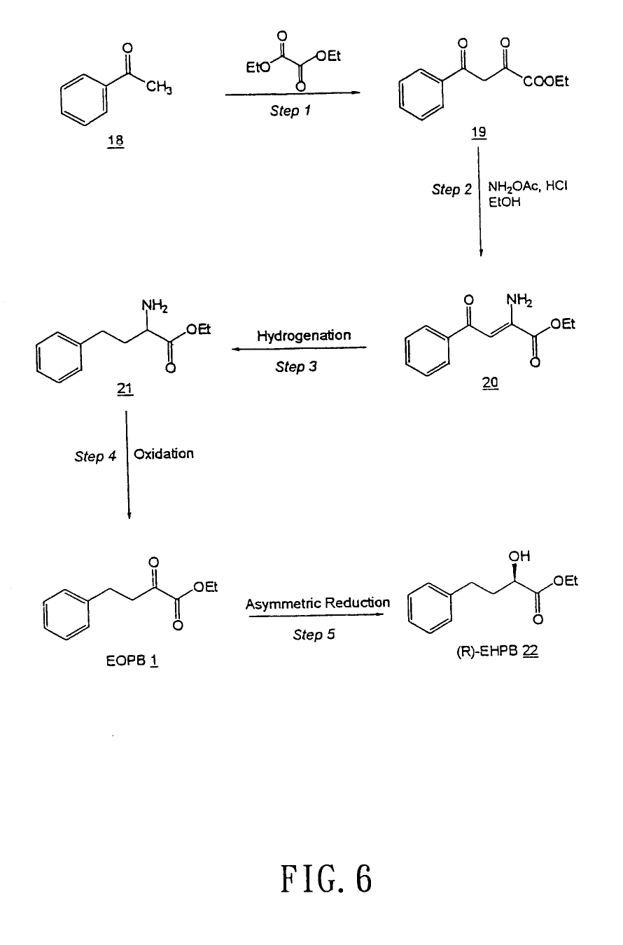

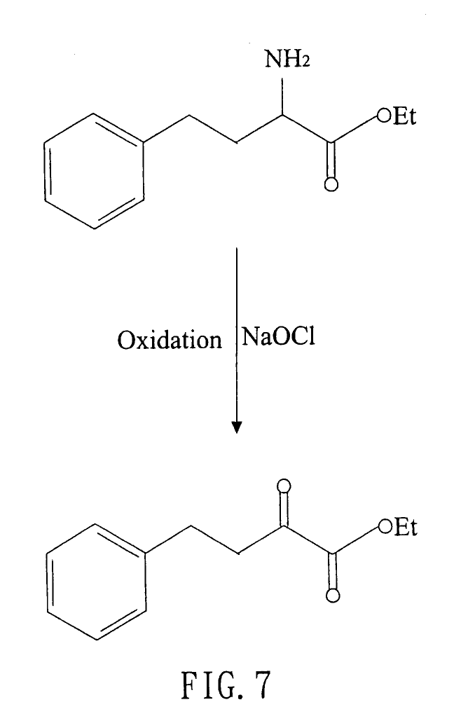

The first embodiment uses Phenylmethyle ketone as initial product. Referring to FIGS. 6 and 7, and comprises the following steps:

Step 1: using material 18 (Phenyl methyl ketone) as initial product and proceeding a condensation process with the Diethyl Oxalate to obtain a compound 19 having diketones;

Step 2: dehumidifying the compound 19 and the acetic acid amine to obtain the compound 20;

Step 3: the compound 20 being treated by hydrogenation process to obtain ethyl hyperphenylalanine compound 21 (Racemic) and to have an amino acid structure. It is to be noted that the amino acid structure in this invention can be obtained from natural amino acid or artificial synthesis amino acid.

Step 4: by proceeding an oxidation reaction of the compound 21 and the sodium hypochlorous acid to have EOPB1 as shown in FIG. 7;

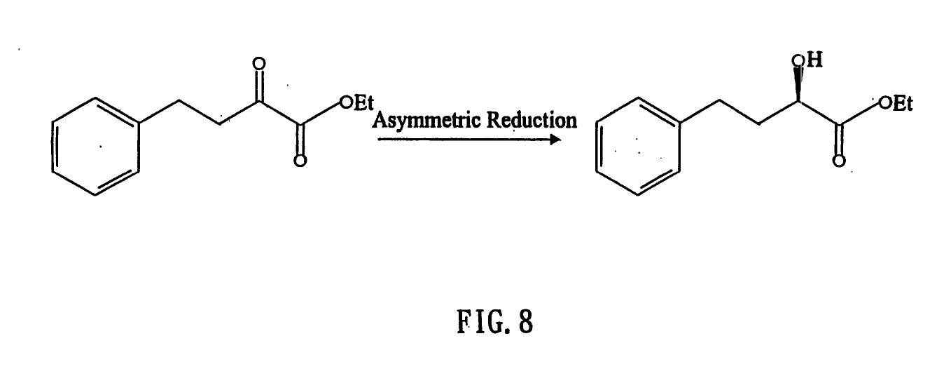

Step 5: the EOPB1 being transferred into (R)-EHPB22 by asymmetric reduction reaction as shown in FIG. 8.

These are the detail steps of the method using the Phenyl methyl ketone as the initial product to obtain EOPB1 and (R)-EHPB22. Figure shows the structure of the ketone acid which is obtained by oxidation reaction of the amino acid and the sodium hypochlorous acid.

In the second embodiment, the LHPE is used as the initial product and the steps of the embodiment are described as follows:

A. putting LHPE, HCl into four-neck distilling flask and adding 4% TBAB, 5% HCl, and a certain amount of EA;

B. reducing the temperature to 0 degree Celsius;

C. adding NaOCl slowly;

D. increasing the temperature to room temperature;

E. heating the bottle till 40 degrees Celsius and tracking the end point of the reaction;

F. reducing the temperature and taking the organic layer of the result combination and removing water and evaporating out the EA;

G. evaporating the final product under 125-135 degrees Celsius and pressure of 2 mm-Hg, and

H. obtaining EOPB1 with the production rate of 84.1% by the above mentioned steps.

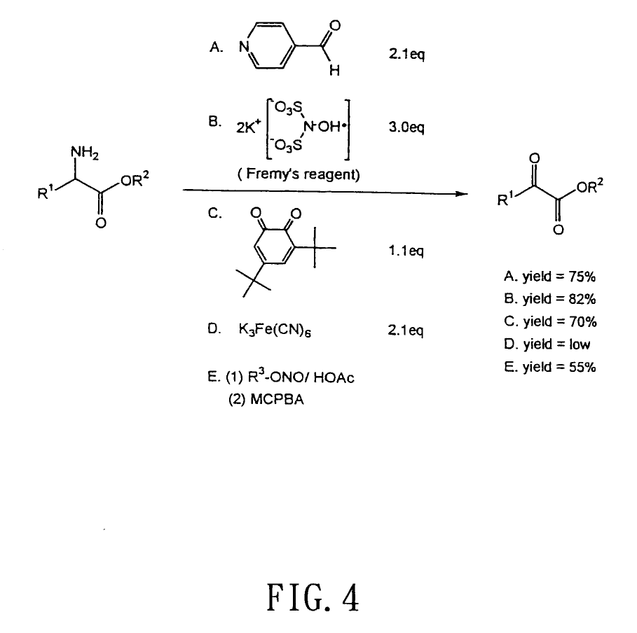

It is to be noted that the present conventional methods using oxidization agent and the amino acid cannot reach the purposes of high production rate and low cost as disclosed in the present invention. As shown in FIG. 4, the first conventional method is a equilibrium reaction which has a low production rate and the separation between final product keto ester and the initial product keto ester is difficult so that it cannot be a method for mass production.

The second convention method as shown in FIG. 4 lists a lot of examples of ratio between the oxidization agent and amino acid. Although these examples have a acceptable rate of production, the price and the amount required of the oxidization agent are too high. In other words, the conventional methods cannot meet the practical needs.

The present invention transfers the amino acid or its derivatives into keto ester by oxidation reaction directly. The temperature is required at the room temperature and the reaction transferring rate is higher than 99%. The method of the present invention obtains the final product at low cost and high production rate, the reaction is stable.

As shown in FIG. 5, the substituents R and X of the amino acid and the ketone acid can be combined with pre-determined compounds to be the derivatives of amino acid or ketone acid, which are shown in the following table:

| R | | X |
|---|---|---|
| CH₃ | (Alanine) | OH |
| CH(CH₃)(C₂H₅) | (Isoleucine) | NH2 |
| CH₂-OOH | (Phenyl alanine) | |
| CH(CH₃)2 | (Valine) | |
| CH₂-CH₂-OOH | (Homophenyl alanine) | |
| PH | (Phenyl glycine) | |

While we have shown and described the embodiment in accordance with the present invention, it should be clear to those skilled in the art that further embodiments may be made without departing from the scope of the present invention.

What is claimed is:

1. A method for transferring amino acid into the ketone acid (ester), the method comprising an oxidation reaction for the amino acid or its derivatives having the structure (I) 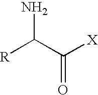
and sodium hypochlorous acid to obtain ketone acid or its derivatives having the structure (II).

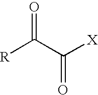

2. The method as claimed in claim 1, wherein the amino acid is obtained by taking Phenyl methyl ketone as initial product which is reacted with the Diethyl Oxalate by condensation reaction to obtain a compound having diketones, the compound being reacted with acetic acid amino by dehumidifying and hydrogenating to obtain the amino acid.

3. The method as claimed in claim 1, wherein the Phenyl methyl ketone is used as initial product and the method comprises the steps:
step 1: using material 18 (Phenyl methyl ketone) as initial product and proceeding a condensation process with the Diethyl Oxalate to obtain a compound having diketones;
step 2: dehumidifying the compound 19 and the acetic acid amine to obtain the compound 20;
step 3: the compound 20 being treated by hydrogenation process to obtain ethyl hyperphenylalanine compound 21 (Racemic) and to have an amino acid structure, the amino acid structure can be obtained from natural amino acid or artificial synthesis amino acid;
step 4: by proceeding an oxidation reaction of the compound 21 and the sodium hypochlorous acid to have EOPB1 as shown below;

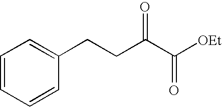

4. The method as claimed in claim 3 wherein the EOPB1 is transferred into derivatives of ketone acid (R)-EHPB22 by asymmetric reduction reaction as shown below:

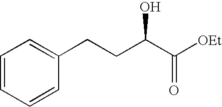

5. The method as claimed in claim 1 wherein the LHPE is used as initial product and the method comprises the steps:
A. putting LHPE, HCl into four neck distilling flask and adding 4% TBAB, 5% HCl, and a certain amount of EA;
B. reducing the temperature to 0 degree Celsius;
C. adding NaOCl slowly;
D. increasing the temperature to room temperature;
E. heating the bottle till 40 degrees Celsius and tracking the end point of the reaction;
F. reducing the temperature and taking the organic layer of the result combination and removing water and evaporating out the EA;
G. evaporating the final product under 125-135 degrees Celsius and pressure of 2 mm-Hg, and
H. obtaining EOPB1 with the production rate of 84.1% by the above mentioned steps.

6. The method as claimed in claim 1, wherein the substituent R of the structure I and structure II is CH3 (Alanine).

7. The method as claimed in claim 1, wherein the substituent R of the structure I and structure II is CH(CH3)(C2H5) (Isoleucine).

8. The method as claimed in claim 1, wherein the substituent R of the structure I and structure II is CH2—OOH (Phenyl alanine).

9. The method as claimed in claim 1, wherein the substituent R of the structure I and structure II is CH(CH3)2(Valine).

10. The method as claimed in claim 1, wherein the substituent R of the structure I and structure II is CH2—CH2—OOH (Homophenyl alanine).

11. The method as claimed in claim 1, wherein the substituent R of the structure I and structure II is PH (Phenylglycine).

12. The method as claimed in claim 1, wherein the substituent X of the structure I and structure II is OH.

13. The method as claimed in claim 1, wherein the substituent X of the structure I and structure II is NH2.
